(19) United States
(12) Patent Application Publication
(10) Pub. No.: US 2025/0263947 A1
(43) Pub. Date: Aug. 21, 2025

(54) METHOD FOR DETERMINING FAILURE CAUSE OF SALT CHLORINATOR FOR SWIMMING POOL

(71) Applicants: Zefeng Chen, Zhejiang (CN); Peifeng Chen, Zhejiang (CN)

(72) Inventors: Yiwu Xu, Zhejiang (CN); Zefeng Chen, Zhejiang (CN); Peifeng Chen, Zhejiang (CN)

(73) Assignees: Zefeng Chen, Zhejiang (CN); Peifeng Chen, Zhejiang (CN)

(21) Appl. No.: 18/857,194

(22) PCT Filed: Jun. 26, 2023
(86) PCT No.: PCT/CN2023/102251

§ 371 Date: Oct. 16, 2024

(30) Foreign Application Priority Data
Dec. 19, 2022 (CN) 202211634180.X

(51) Int. Cl. E04H 4/14; G01N 33/18

(52) U.S. Cl. CPC E04H 4/14; G01N 33/1853

(57) ABSTRACT

The present invention provides a method for determining a failure cause of a salt chlorinator for a swimming pool, includes: determining whether a water quality test report of swimming pool water can be provided; and if not, proceeding: determining whether there is a problem with the salt chlorinator, where the determining whether there is a problem with the salt chlorinator includes determining whether there is a posed problem and/or determining electrode life, includes: determining whether there is the posed problem; performing calculation based on a posed problem determining formula and determining whether a value thereof is greater than 0; and if not, outputting a first alarm signal; and determining electrode life; performing calculation based on an electrode life determining formula, and determining whether a value is less than a threshold A; and if not, determining that the electrode life is to be exhausted, and outputting a second alarm signal.

### FIELD OF TECHNOLOGY

The present invention relates to the field of salt chlorinators for swimming pools, and in particular to a method for determining a failure cause of a salt chlorinator for a swimming pool.

### BACKGROUND

In swimming pools as public activity places, human secretions infiltrate into water during swimming, which causes the breeding of bacteria and algae microorganisms, affecting the water quality. Therefore, the water needs to be disinfected. Hypochlorous acid is a strong oxidant that can kill bacteria in the water, which is often used to sterilize and disinfect swimming pool water. At present, the hypochlorous acid is mainly prepared in the following way: dilute hydrochloric acid (sodium chloride salt solution) is electrolyzed into chlorine gas and hydrogen gas through an electrolysis apparatus, and then the chlorine gas is dissolved in water to generate the hypochlorous acid. A salt chlorinator is a device that performs sterilization and disinfection by electrolyzing the dilute hydrochloric acid (sodium chloride salt solution).

A buyer and a seller usually conduct technical exchange through the Internet in case of an after-sales problem with the salt chlorinator for a swimming pool. Both the buyer and seller lack technologies, it is difficult to ask specialized engineers for help, there is a serious lack of staffing, and there is a time difference during communication. When there is a problem with the device, there are many causes, and it is difficult to quickly find the problem, which brings inconvenience to both the buyer and seller. Moreover, in practice, the impact of water quality is usually ignored, so that the problem cannot be comprehensively determined. Therefore, a complete and standardized method for determining a failure cause of the salt chlorinator needs to be designed.

### SUMMARY

### (1) A Technical Problem to be Resolved

A problem to be resolved in the present invention is to provide a method for determining a failure cause of a salt chlorinator for a swimming pool, to overcome defects of a difficulty in locating a failure cause of an existing salt chlorinator and insufficient after-sales technical force.

### (2) Technical Solutions

To resolve the technical problem, the present invention provides a method for determining a failure cause of a salt chlorinator for a swimming pool. The method includes the following steps:
step S1: determining whether a water quality test report of swimming pool water can be provided; and
if yes, proceeding to step S2: determining whether there is a quality problem with the swimming pool water;
if not, proceeding to step S3: determining whether there is a problem with the salt chlorinator,
wherein the determining whether there is a problem with the salt chlorinator comprises determining whether there is a posed problem and/or determining electrode life.

Further, wherein step S3 comprises the following steps:
step S31: determining whether there is the posed problem; based on a percentage Pec of a chlorine production yield and actual working time t set by a user, performing calculation based on a posed problem determining formula and determining whether a value thereof is greater than 0; and
if yes, determining whether there is other problem with the salt chlorinator;
if not, outputting a first alarm signal;
step S32: determining electrode life; based on an actual salt concentration Fₙ of the swimming pool and a current I and voltage U of an electrode, performing calculation based on an electrode life determining formula f(Fₙ, I, U) and determining whether a value thereof is less than a threshold A;
if yes, determining that the electrode life is not exhausted, and determining whether there is other problem with the salt chlorinator;
if not, determining that the electrode life is to be exhausted, and outputting a second alarm signal.

Further, wherein the posed problem determining formula is specifically as follows:

$$f ( I , Pec , t ) = {K}_{1} \times {K}_{4} \times I \times Pec \times t - {ppm}_{s t a n d a r d}$$

wherein K₁ is a coefficient of a serial-parallel connection mode corresponding to an electrode model, K₄ is a reciprocal of a maximum volume of the swimming pool that can match the electrode model, I is the current of the electrode, Pec is a percentage of a chlorine production yield of the device, t is the actual working time of the device, and ppm_{standard} is a standard value of residual chlorine.

Further, wherein the posed problem determining formula is specifically as follows:

$$f ( m , Pec , t ) = {K}_{4} \times m \times Pec \times t - {ppm}_{s t a n d a r d}$$

wherein K₄ is a reciprocal of a maximum volume of the swimming pool that can match an electrode model, m is the number of grams of chlorine per hour corresponding to the electrode model, Pec is a percentage of a chlorine production yield of the device, t is the actual working time of the device; and ppm_{standard} is a standard value of residual chlorine.

Further, wherein the electrode life determining formula is specifically as follows:

$$f ( {F}_{n} , I , U ) = {F}_{n} - {K}_{2} \times {K}_{3} \times \frac{I}{{U}^{n}}$$

wherein Fₙ is an actual salt concentration in the swimming pool, K₂ is an electrode constant corresponding to an electrode model, K₃ is a temperature compensation coefficient, I is the current of the electrode, U is the voltage of the electrode, n is a constant value greater than 0, and A is within 300-1,200.

Further, wherein the electrode life determining formula is specifically as follows:

$$f ( {F}_{n} , I , U ) = 1 - \frac{{K}_{2} \times {K}_{3} \times I}{{U}^{n} \times {F}_{n}}$$

wherein Fₙ is an actual salt concentration in the swimming pool, K₂ is an electrode constant corresponding to an electrode model. K₃ is a temperature compensation coefficient, I is the current of the electrode, U is the voltage of the electrode, n is a constant value greater than 0, and A is within 10%-30%.

Further, wherein after the outputting a first alarm signal, the following step is performed:
step S311: cleaning the electrode; using acidic solution to clean the electrode to remove dirt on a surface of the electrode; and after cleaning, repeat step S31, and if it is determined that the value is still less than 0, proceeding to step S32.

Further, wherein before step S31, the following step is performed:
step S301: determining V; based on an actual volume V of the swimming pool, obtaining a model of a currently working electrode, looking up a used volume threshold V_{threshold} corresponding to the electrode model, and determining whether V is less than or equal to V_{threshold};
if yes, determining that the electrode model is selected properly, and proceeding to step S302;
if not, determining that the electrode model is not selected properly, and outputting a third alarm signal;

step S302: determining Pec and t; obtaining a percentage Pec of a chlorine production yield and actual working time t set by the user, and performing calculation based on f(Pec, t)=Pec×t and determining whether a value thereof is greater than 1; and
if yes, determining that Pec and t are set properly, and proceeding to step S31;
if not, determining that Pec and t are not set properly, and outputting a fourth alarm signal.

Further, wherein before step S32, the following step is performed:
step S312: determining Fₙ, detecting an actual salt concentration Fₙ in the swimming pool, and then determining whether 2,300 ppm≤Fₙ≤6,000 ppm is established; and
if yes, determining that Fₙ added by the user is proper, and proceeding to step S32;
if not, determining that Fₙ added by the user is not proper, and outputting a fifth alarm signal.

Further, wherein step S2 is specifically: based on numerical values of a total phosphorus concentration C_{P} in the swimming pool, a cyanuric acid concentration C_{y} in the swimming pool, pH of swimming pool water, a calcium hardness concentration Ch in the swimming pool, and a total alkali concentration Ct in the swimming pool in the water quality test report, determining whether any one or more of C_{P}≤C_{Pthreshold}, C_{y1}≤C_{y}≤C_{y2}, pH₁≤pH≤pH₂, Ch₁≤Ch≤Ch₂, and Ct₁≤Ct≤Ct₂ are established; and
if yes, determining that there is no problem with water quality, and proceeding to step S3 to continuously determine whether there is a problem with the salt chlorinator;
if not, determining that there is a problem with water quality, and outputting a sixth alarm signal.

Further, wherein step S3 comprises the following steps:
determining electrode life; based on an actual salt concentration Fₙ in the swimming pool and a current I and voltage U of an electrode, performing calculation based on an electrode life determining formula f(Fₙ, I, U) and determining whether a value thereof is less than a threshold A;
if yes, determining that the electrode life is not exhausted, and determining whether there is other problem with the salt chlorinator;
if not, determining that the electrode life is to be exhausted, and outputting a second alarm signal;
determining whether there is the posed problem; based on a percentage Pec of a chlorine production yield, actual working time t, and the current I of the electrode set by a user, performing calculation based on a posed problem determining formula f(I, Pec, t) and determining whether a value thereof is greater than 0;
if yes, determining whether there is other problem with the salt chlorinator; or
if not, outputting a first alarm signal.

Further, step S3 further includes the following step:
step S33: determining whether there is a problem with a temperature sensor; detecting the temperature sensor, and if there is a problem with the temperature sensor, outputting a seventh alarm signal, or if there is no problem with the temperature sensor, outputting an eighth alarm signal.

### (3) Beneficial Effects

In standard and comprehensive steps of the method for determining a failure cause of a salt chlorinator for a swimming pool provided in the present invention, the failure cause of the salt chlorinator can be determined step by step, and a complete detection process is provided, so that the failure cause can be found accurately and rapidly, and a corresponding failure signal is output to help the user process the failure. Determining is running only by inputting a corresponding parameter based on a prompt of the system, and the user or after-sales personnel does not need to understand professional and technical knowledge, which saves communication time and reduces after-sales steps. This method can be built into the salt chlorinator, programmed into a website, built into a mobile device, or the like, which is easily operated and used. In addition, a standard process for determining water quality is provided, to determine a water quality problem that is easily ignored in after-sales, find out which specific parameter in water quality has a problem, and resolve an after-sales problem more accurately and comprehensively, which overcomes defects of insufficient after-sales technical strength for an existing salt chlorinator and a difficulty in locating the failure cause.

### BRIEF DESCRIPTION OF THE DRAWINGS

FIG. 1 is a schematic flow chart of a method for determining a failure cause of a salt chlorinator for a swimming pool according to a first embodiment;

FIG. 2 is a schematic flow chart of specific steps of the method for determining a failure cause of a salt chlorinator for a swimming pool according to the first embodiment;

FIG. 3 is a schematic flow chart of a method for determining a failure cause of a salt chlorinator for a swimming pool according to a second embodiment; and

FIG. 4 is a schematic flow chart of a method for determining a failure cause of a salt chlorinator for a swimming pool according to a fifth embodiment.

### DESCRIPTION OF THE EMBODIMENTS

Specific implementations of the present invention are further described in detail with reference to accompanying drawings and embodiments. The following embodiments are used to describe the present invention, but are not used to limit the scope of the present invention.

### First Embodiment

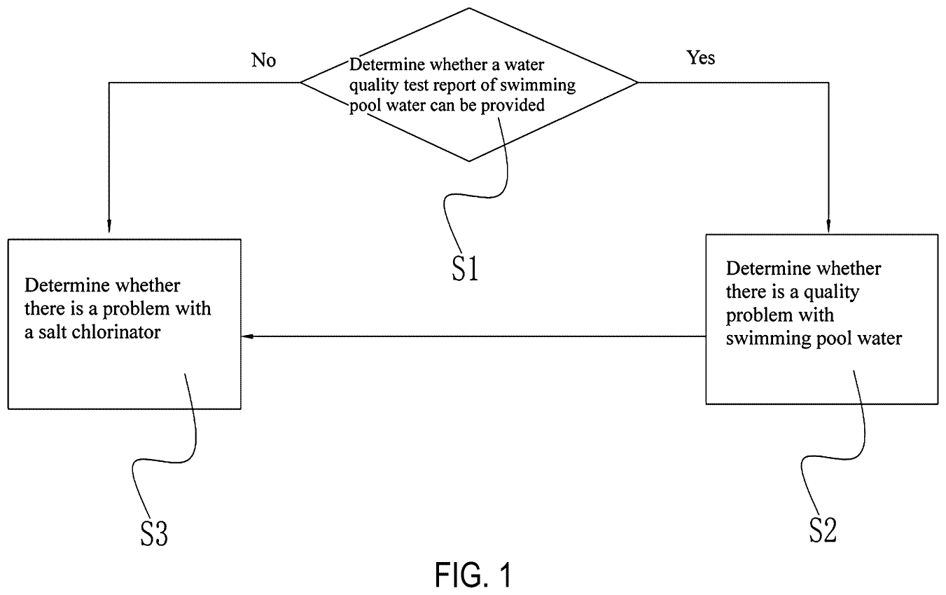

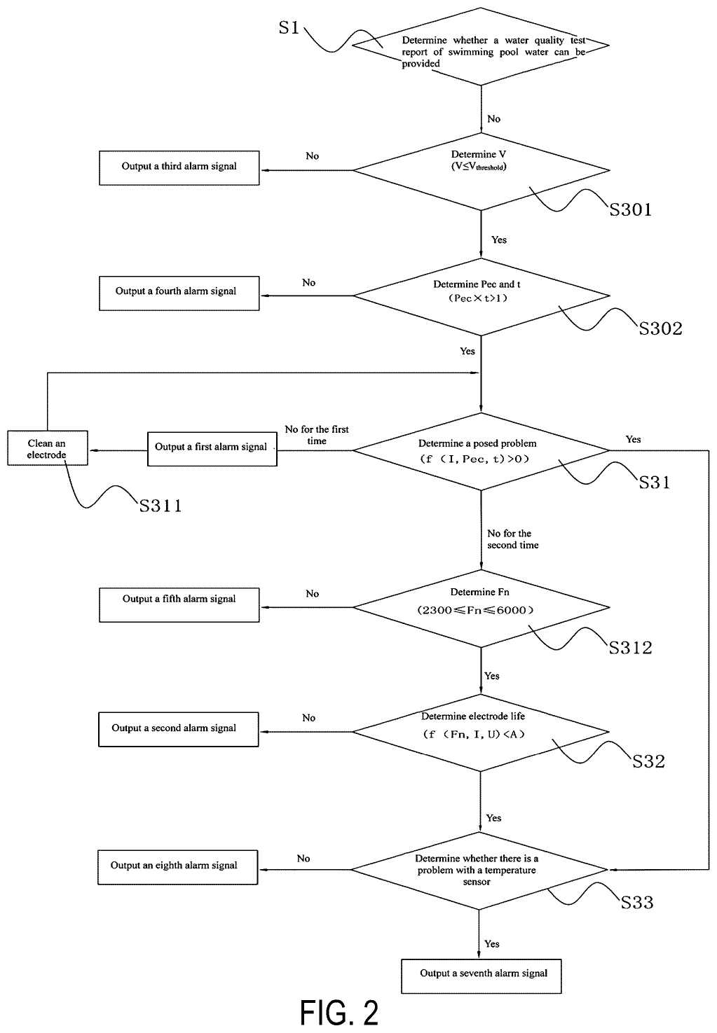

Refer to FIG. 1 and FIG. 2. This embodiment provides a method for determining a failure cause of a salt chlorinator for a swimming pool, including the following steps:
step S1: ask, by a system, whether a water quality test report of swimming pool water can be provided; and
if yes, proceed to step S2: determine whether there is a quality problem with the swimming pool water;
if not, proceed to step S3: determining whether there is a problem with the salt chlorinator,
where the determining whether there is a problem with the salt chlorinator includes determining whether there is a posed problem and/or determining electrode life.

Based on a frequency at which the failure cause occurs, there is usually no problem with the water quality of the swimming pool water, and it is also difficult for a user to obtain the water quality test report of swimming pool water, so that it is difficult to provide the water quality test report. Therefore, step S3: determining whether there is a problem with the salt chlorinator is directly performed.

Refer to FIG. 2. In this embodiment, step S3 specifically includes the following steps.
step S301: determine V. The user enters an actual volume V of the swimming pool based on a system instruction, the system obtains a model of a currently working electrode, and looks up Table 1 preset in the system to find a used volume threshold V_{threshold} corresponding to the electrode model and determine whether V is less than or equal to V_{threshold}. V_{threshold} is a volume threshold determined by a manufacturer based on a product model of the manufacturer, that is, a maximum value of a volume of the swimming pool corresponding to this model. For example, the system obtains the electrode model as model I, and it is obtained by looking up the table that V_{threshold} is 20,000 US gallons.

If yes, determine that the electrode model is selected properly, and proceed to step S302.

If not, determine that the electrode model is not selected properly, and output a third alarm signal. The third alarm signal may be “The electrode model is not selected properly, please select an electrode with a proper model.”

When purchasing the salt chlorinator, the user may purchase a chlorinator of a small model or mistakenly buy a chlorinator of another model to save costs. Through step S301, it can be determined whether the electrode model purchased by the user is proper.
Step S302: determine Pec and t. The system obtains a percentage Pec of a chlorine production yield and actual working time t set by the user, performs calculation based on f(Pec, t)=Pec×t and determines whether a value thereof is greater than 1. Table 2 shows Pec and t set to be corresponding to volumes of common swimming pools, which can be used for reference.

If yes, determine that Pec and t are set properly, and proceed to step S31.

If not, determine that Pec and t are not set properly, and output a fourth alarm signal. The fourth alarm signal may be “Pec and t are not set properly, please increase Pec and t.”

After purchasing, the user may not be proficient in use of the salt chlorinator, the user sets the percentage Pec of a chlorine production yield the actual working time t to small values, and the chlorine production yield of the salt chlorinator is limited, so that the use effect is poor. Through step S302, it can be determined whether Pec and t set by the user are proper.
Step S31: determine whether there is a posed problem. The system obtains the percentage Pec of a chlorine production yield and actual working time t set by the user, detects a current I of an electrode by using a current collection circuit in the device, and then performs calculation based on a posed problem determining formula and determines whether a value is greater than 0.

If yes, determine whether there is other problem with the salt chlorinator. For example, proceed to step S33.

If not, output a first alarm signal. The first alarm signal may be “There is a problem with the electrode, please clean the electrode.”

In the salt chlorinator device, the electrode is a main working component. When there is a failure in the device, there is probably a failure in the electrode. Through step S31, it can be determined whether there is a problem with the electrode. When it is determined that the value is less 0, it can be determined that the electrode needs to be repaired.

Specifically, the posed problem determining formula is as follows:

$$f ( I , Pec , t ) = {K}_{1} \times {K}_{4} \times I \times Pec \times t - {ppm}_{s t a n d a r d}$$

where K₁ is a coefficient of a serial-parallel connection mode corresponding to the electrode model, and a value K₁ corresponding to the electrode model is found by looking up Table 1 preset in the system. For example, the electrode model is model I, and it is obtained by looking up the table that K₁ is 6; K₄ is a reciprocal of an actual volume of the swimming pool, that is,

$${K}_{4} = \frac{1}{V} ;$$

I is a current of the electrode, Pec is a percentage of a chlorine production yield of the device, t is the actual working time of the device; and ppm_{standard} is a standard value of residual chlorine, and ppm_{standard} is 1 ppm.
Step S311: clean the electrode; shut down the device, disassemble the electrode, and use acidic solution to clean the electrode to remove dirt on a surface of the electrode; and after cleaning, assemble the electrode to the device, restart the device and repeat step S31, and if it is determined that the value is still less than 0, proceed to step S32.

When the electrode is used in electrolysis, dirt is easily generated on the electrode, and the dirt affects the use effect of the electrode. Through step S311, the electrode is cleaned to remove the dirt on the surface and prevent the dirt from affecting the chlorine production effect. After cleaning, if it is determined that the value is still less than 0 through step S31, it indicates that the removal of dirt cannot resolve the problem, and then it is determined whether there is a problem with electrode life.
Step S312: Determine Fₙ. The system detects an actual salt concentration Fₙ in the swimming pool by using a salinization meter in the device, and then determines whether 2,300 ppm≤Fₙ≤6,000 ppm is established.

If yes, determine that Fₙ added by the user is proper, and proceed to step S32.

If not, determine that Fₙ added by the user is not proper, and output a fifth alarm signal. The fifth alarm signal may be “Please add hydrochloric acid (sodium chloride salt) solution at a proper concentration.”

When the salt chlorinator is used, the user needs to add the hydrochloric acid (sodium chloride salt) solution at a proper concentration, and the user may mistakenly add hydrochloric acid (sodium chloride salt) solution at another concentration due to unskilled use, which affects the use effect. Through step S312, it can be determined whether the concentration of the hydrochloric acid (sodium chloride salt) solution added by the user is proper.
Step S32: determine electrode life; and based on the actual salt concentration Fₙ of the swimming pool, detect a current I of the electrode by using the current collection circuit, detect a voltage U of the electrode by using the voltage collection circuit, and then perform calculation through an electrode life determining formula f(Fₙ, I, U) and determine whether a value is less than a threshold A.

If yes, determine that the electrode life is not exhausted, and determine whether there is other problem with the salt chlorinator. For example, proceed to step S33.

If not, determine that the electrode life is to be exhausted, and output a second alarm signal. The second alarm signal may be “The electrode life is to be exhausted, please replace the electrode.”

Through step S31, it can be determined that the electrode needs to be repaired. For the dirt and life problems in the electrode, dirt on the electrode is removed through step S311. If the failure problem is not resolved, it is determined whether there is a life problem. Through step S32, it can be determined whether there is an electrode life problem.

Specifically, the electrode life determining formula is as follows:

$$f ( {F}_{n} , I , U ) = {F}_{n} - {K}_{2} \times {K}_{3} \times \frac{I}{{U}^{n}}$$

where Fₙ is the actual salt concentration in the swimming pool; K₂ is an electrode constant corresponding to the electrode model, a value of K₂ can be obtained by looking up Table 1 preset in the system, for example, the electrode model is electrode model I, and it is obtained by looking up the table that K₂ is 21,700,000; K₃ is a temperature compensation coefficient, I is the current of the electrode, U is the voltage of the electrode, n is a constant value greater than 0, and n is preferably 3; and A is within 300-1,200, preferably 500.

Further, K₃ is determined by a relation about T:

$${K}_{3} = f ( T ) = \frac{75 - T}{50}$$

where T is a temperature of water flowing through the electrode, and T can be measured by a temperature sensor in the device in degrees Celsius (° C.).
Step S33: determine whether there is a problem with the temperature sensor; use a multimeter to detect the temperature sensor, and if there is a problem with the temperature sensor, output a seventh alarm signal, or if there is no problem with the temperature sensor, output an eighth alarm signal. The seventh alarm signal may be “There is a problem with the temperature sensor, please replace the temperature sensor”, and the eighth alarm signal may be “There is a problem with the motherboard, please detect the motherboard.”

Through steps before step S33, common failures in the salt chlorinator are removed. If no cause is found, it is determined whether there is a problem with the temperature sensor or the motherboard through step S33. The determining whether there is a problem with the temperature or the motherboard is performed at last because there is usually no problem with the temperature sensor or the motherboard.

The method for determining a failure cause of a salt chlorinator for a swimming pool may be directly built into a host of the salt chlorinator. A display screen and button are set on the host of the salt chlorinator, to help the user input parameters. When a failure occurs, the failure can be determined by direct operation of the salt chlorinator, which is more easily used. Alternatively, the method may be programmed into an after-sales website, and the user performs determining through the after-sales website. Some of parameters that need to be obtained from the device can be obtained through communication between the Internet of Things and the website. Alternatively, this method can be written into an app and downloaded to a mobile device, such as a mobile phone or tablet, and some of the parameters that need to be obtained from the device can be obtained through communication between the Internet of Things and a mobile device.

TABLE 1
V_{threshold}, K₁, and K₂ corresponding to electrode models

| | V_{threshold} | | | m |
|---|---|---|---|---|
| Electrode model | (US gallons) | K₂ | K₁ | (g/h) |
| Electrode model I | 20,000 | 21,700,000 | 6 | 20 |
| Electrode model II | 30,000 | 15,400,000 | 6 | 30 |
| Electrode model III | 40,000 | 11,400,000 | 6 | 40 |
| Electrode model IV | 60,000 | 7,800,000 | 6 | 60 |

TABLE 2
Setting of Pec and t corresponding
to common swimming pool volume

| V (m³) | Pec (%) | T (h) |
|---|---|---|
| 76 | 100 | 8 |
| 114 | 50 | 16 |
| 151 | 80 | 10 |
| 227 | 100 | 8 |

TABLE 3
Meanings corresponding to alarm signals

| Alarm signal | Meaning |
|---|---|
| First alarm signal | There is a problem with the |
| | electrode, please clean the electrode |
| Second alarm signal | The electrode life is to be |
| | exhausted, please replace the electrode |
| Third alarm signal | The electrode model is not selected |
| | properly, please select an |
| | electrode with a proper model |
| Fourth alarm signal | Pec and t are not set properly, |
| | please increase Pec and t |
| Fifth alarm signal | Please add hydrochloric acid |
| | (sodium chloride salt) solution at a |
| | proper concentration |
| Sixth alarm signal | There is a water quality problem, |
| | please improve the water quality |
| Seventh alarm signal | There is a problem with the |
| | temperature sensor, please replace the |
| | temperature sensor |
| Eighth alarm signal | There is a problem with the |
| | motherboard, please detect the |
| | motherboard |

### Second Embodiment

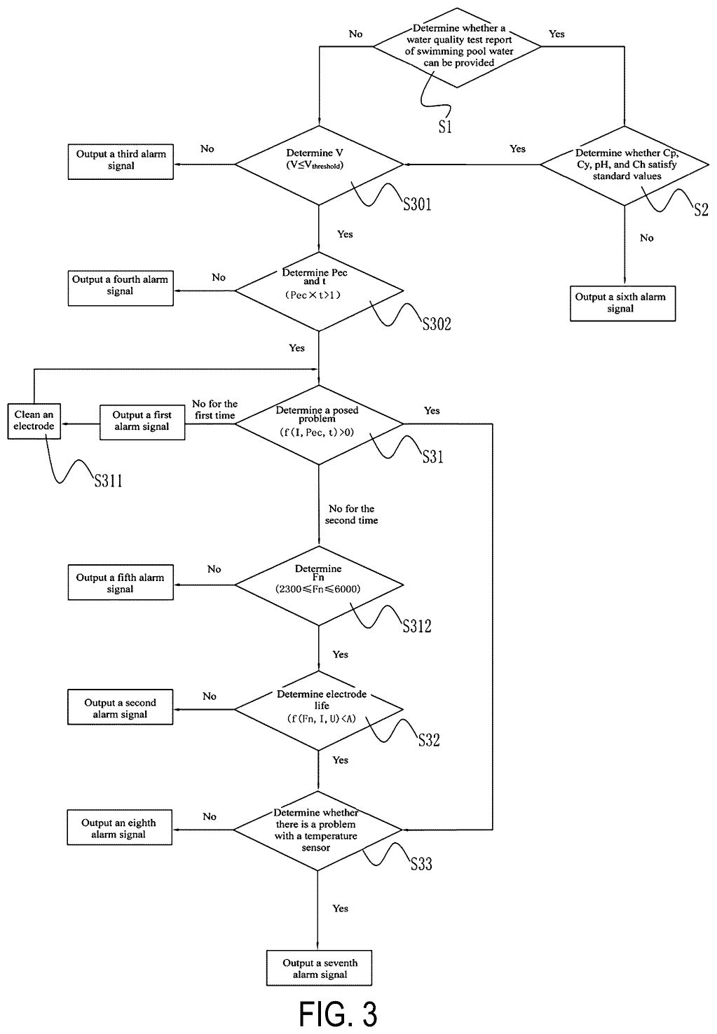

Refer to FIG. 3. This embodiment provides a method for determining a failure cause of a salt chlorinator for a swimming pool, which is different from the method in the first embodiment as follows: in this embodiment, a user can provide a water quality test report of swimming pool water, and determine whether there is a problem with water quality of the swimming pool in step S2.

Step S2 is specifically as follows: based on numerical values of a total phosphorus concentration C_{P} in the swimming pool, a cyanuric acid concentration C_{y} in the swimming pool, pH of swimming pool water, a calcium hardness concentration Ch in the swimming pool, and a total alkali concentration Ct in the swimming pool in the water quality test report, determine whether all or one or more of C_{P}≤C_{Pthreshold}, C_{y1}≤C_{y}≤C_{y2}, pH₁≤pH≤pH₂. Ch₁≤Ch≤Ch₂, and Ct₁≤Ct≤Ct₂ are established, leading to slightly worse use effect. As C_{P} is higher, nutrients are provided for rapid growth of bacteria and algae, and chlorine in the water is quickly consumed, and C_{Pthreshold} may be 100 ppb. As C_{y} is lower, chlorine is quickly broken down by sunlight. As C_{y} is higher, efficacy of chlorine on algae and bacteria is reduced, C_{y} is ideally 30-50 ppm, C_{y1} may be 30 ppm, and C_{y1} is 100 ppm. As a pH value is extremely low, the chlorine dissipates quickly. As the pH value is extremely high, disinfection efficiency of a disinfectant is low. The pH value is ideally 7.4-7.6, pH₁ is 7.2, pH₂ is 7.8; Ch₁ is 150 ppm, Ch₂ is 500 ppm: Ct₁ is 80 ppm, and Ct₂ is 120 ppm.

If yes, determine that there is no problem with the water quality, and proceed to step S3 to continuously determine whether there is a problem with the salt chlorinator.

If not, determine that there is a problem with the water quality, and output a sixth alarm signal. The sixth alarm signal may be “There is a problem with the water quality, please improve the water quality”.

In after-sales, the impact of a water quality problem is usually ignored, and a cause for the poor use effect is still not found after all faults are removed from the device. Through step S2, a standard process for determining water quality is formulated to determine whether there is a water quality problem, find out which specific parameter in water quality has a problem, and resolve the after-sales problem more accurately and comprehensively.

### Third Embodiment

A method for determining a failure cause of a salt chlorinator for a swimming pool provided in this embodiment is different from the method in the first embodiment as follows: step S31 is different.

In this embodiment, in step S31, based on a percentage Pec of a chlorine production yield and actual working time t set by the user, and the number of grams m of chlorine per hour corresponding to an electrode model preset in the system, calculation is performed based on a posed problem determining formula, and it is determined whether a value is greater than 0.

Further, the posed problem determining formula is as follows:

$$f ( m , Pec , t ) = {K}_{4} \times m \times P ec \times t - pp {m}_{s t a n d a r d}$$

where K₄ is a reciprocal of an actual volume of the swimming pool, that is,

$${K}_{4} = \frac{1}{V} ; m$$

is the number of grams of chlorine per hour corresponding to the electrode model, and a value m corresponding to the electrode model is found by looking up Table 1 preset in the system. For example, the electrode model is model I, and it is obtained by looking up the table that m is 20 g/h. Pec is a percentage of a chlorine production yield of the device, t is the actual working time of the device, ppm_{standard} is a standard value of residual chlorine, ppm_{standard} may be 1-4 ppm, and ppm_{standard} is 1 in this embodiment.

### Fourth Embodiment

A method for determining a failure cause of a salt chlorinator for a swimming pool provided in this embodiment is different from the method in the first embodiment as follows: in step S32, an electrode life determining formula is different.

In this embodiment, the electrode life determining formula is specifically as follows:

$$f ( {F}_{n} , I , U ) = 1 - \frac{{K}_{2} \times {K}_{3} \times I}{{U}^{n} \times {F}_{n}}$$

where Fₙ is an actual salt concentration in the swimming pool, K₂ is an electrode constant corresponding to an electrode model. K₃ is a temperature compensation coefficient, I is the current of the electrode, U is the voltage of the electrode, n is a constant value greater than 0, n may be 3, and A is within 10%-30%.

### Fifth Embodiment

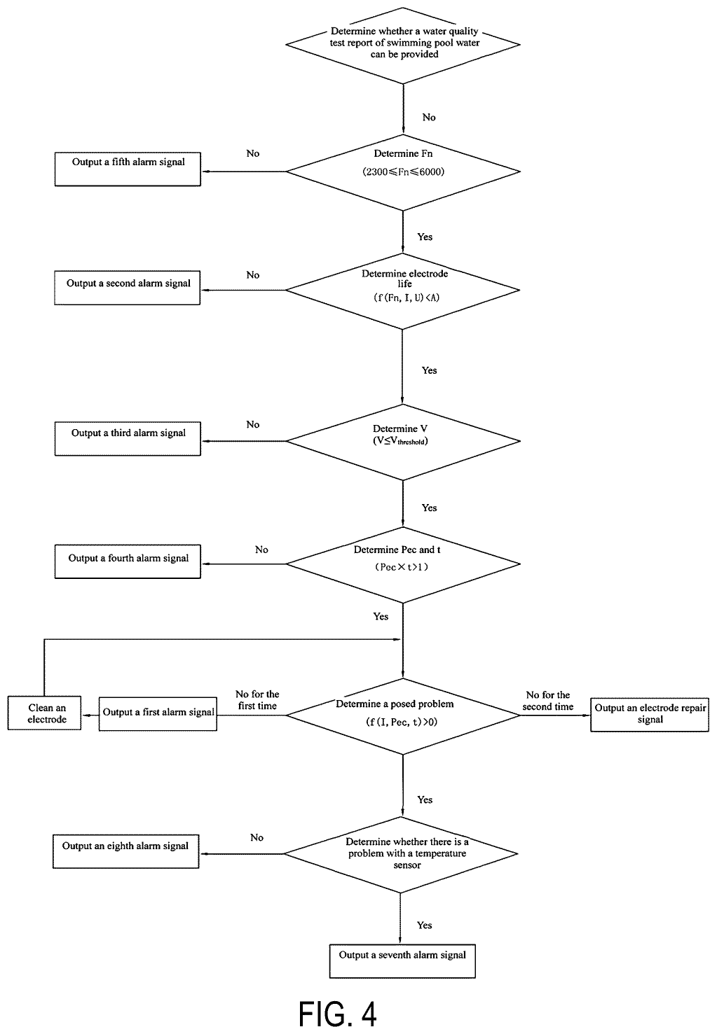

Refer to FIG. 4. A method for determining a failure cause of a salt chlorinator for a swimming pool provided in this embodiment is different from the method in the first embodiment as follows: a sequence of a step for determining electrode life and a step for determining a posed problem is different.

In this embodiment, step S3 includes the following steps.

First, determine Fₙ, with a specific process the same as the process in the first embodiment;
determine electrode life; based on an actual salt concentration Fₙ in the swimming pool and a current I and voltage U of an electrode, perform calculation based on an electrode life determining formula f(Fₙ, I, U) and determine whether a value thereof is less than a threshold A; and
if yes, determine that the electrode life is not exhausted, and determine whether there is a posed problem; or
if not, determine that the electrode life is to be exhausted, and output a second alarm signal. The second alarm signal may be “The electrode life is to be exhausted, please replace the electrode.”

Before determining whether there is a posed problem, determine V, and then determine Pec and t in sequence, with a specific process the same as the process in the first embodiment.

Determine whether there is the posed problem; based on a percentage Pec of a chlorine production yield, actual working time t, and the current I of the electrode set by a user, perform calculation based on a posed problem determining formula f(I, Pec, t) and determine whether a value thereof is greater than 0;
if yes, determine whether there is another problem with the salt chlorinator, where, for example, determine whether there is a problem with a temperature sensor;
if not, output a first alarm signal. The first alarm signal may be “There is a problem with the electrode, please clean the electrode.”

Clean the electrode; shut down the device, disassemble the electrode, and use acidic solution to clean the electrode to remove dirt on a surface of the electrode; and after cleaning, assemble the electrode to the device, restart the device and repeat the posed problem determining step. If it is determined that the value is still less than 0, output an alarm signal “Please repair the electrode”.

In standard and comprehensive steps of the method for determining a failure cause of a salt chlorinator for a swimming pool provided in the present invention, the failure cause of the salt chlorinator can be determined step by step, and a complete detection process is provided, so that the failure cause can be located accurately and rapidly, and a corresponding failure signal is output to help the user process the failure. Determining is running only by inputting a corresponding parameter based on a prompt of the system, and the user or after-sales personnel does not need to understand professional and technical knowledge, which saves communication time and reduces after-sales steps. This method can be built into the salt chlorinator, programmed into a website, built into a mobile device, or the like, which is easily operated and used. In addition, a standard process for determining water quality is provided, to determine a water quality problem that is easily ignored in after-sales, find out which specific parameter in water quality has a problem, and resolve an after-sales problem more accurately and comprehensively, which overcomes defects of insufficient after-sales technical strength for an existing salt chlorinator and a difficulty in locating the failure cause.

The foregoing description is preferred implementations of the present invention. It should be noted that those of ordinary skill in the art can make various improvements and modifications without departing from the technical principles of the present invention, which shall fall within the protective scope of the present invention.

1. A method for determining a failure cause of a salt chlorinator for a swimming pool, comprising the following steps:
step S1: determining whether a water quality test report of swimming pool water can be provided; and
if the water quality test report of the swimming pool water can be provided, proceeding to step S2: determining whether there is a quality problem with the swimming pool water; or
if the water quality test report of the swimming pool water cannot be provided, proceeding to step S3: determining whether there is a problem with the salt chlorinator,
wherein the determining whether there is a problem with the salt chlorinator comprises determining whether there is a posed problem and/or determining electrode life.

2. The method for determining a failure cause of a salt chlorinator for a swimming pool according to claim 1, wherein the step S3 comprises the following steps:
step S31: determining whether there is the posed problem; based on a percentage Pec of a chlorine production yield and actual working time t set by a user, performing calculation based on a posed problem determining formula and determining whether a value thereof is greater than 0; and
if the value is greater than 0, determining whether there is other problem with the salt chlorinator; or
if the value is not greater than 0, outputting a first alarm signal; and
step S32: determining electrode life; based on an actual salt concentration Fₙ of the swimming pool and a current I and voltage U of an electrode, performing calculation based on an electrode life determining formula f(Fₙ, I, U) and determining whether a value thereof is less than a threshold A; and
if the value is less than the threshold A, determining that the electrode life is not exhausted, determining whether there is other problem with the salt chlorinator;
if the value is not less than the threshold A, determining that the electrode life is to be exhausted, outputting a second alarm signal.

3. The method for determining a failure cause of a salt chlorinator for a swimming pool according to claim 2, wherein the posed problem determining formula is specifically as follows:
$$f ( I , Pec , t ) = {K}_{1} \times {K}_{4} \times I \times Pec \times t - {ppm}_{s t a ndard}$$
wherein K₁ is a coefficient of a serial-parallel connection mode corresponding to an electrode model, K₄ is a reciprocal of a maximum volume of the swimming pool that can match the electrode model, I is the current of the electrode, Pec is a percentage of a chlorine production yield of the device, t is the actual working time of the device, and ppm_{standard} is a standard value of residual chlorine.

4. The method for determining a failure cause of a salt chlorinator for a swimming pool according to claim 2, wherein the posed problem determining formula is specifically as follows:
$$f ( m , Pec , t ) = {K}_{4} \times m \times P ec \times t - {ppm}_{s tandard}$$
wherein K₄ is a reciprocal of a maximum volume of the swimming pool that can match an electrode model, m is the number of grams of chlorine per hour corresponding to the electrode model, Pec is a percentage of a chlorine production yield of the device, t is the actual working time of the device; and ppm_{standard} is a standard value of residual chlorine.

5. The method for determining a failure cause of a salt chlorinator for a swimming pool according to claim 2, wherein the electrode life determining formula is specifically as follows:
$$f ( {F}_{n} , I , U ) = {F}_{n} - {K}_{2} \times {K}_{3} \times \frac{I}{{U}^{n}}$$
wherein Fₙ is an actual salt concentration in the swimming pool, K₂ is an electrode constant corresponding to an electrode model, K₃ is a temperature compensation coefficient, I is the current of the electrode, U is the voltage of the electrode, n is a constant value greater than 0, and A is within 300-1,200.

6. The method for determining a failure cause of a salt chlorinator for a swimming pool according to claim 2, wherein the electrode life determining formula is specifically as follows:
$$f ( {F}_{n} , I , U ) = 1 - \frac{{K}_{2} \times {K}_{3} \times I}{{U}^{n} \times {F}_{n}}$$
wherein Fₙ is an actual salt concentration in the swimming pool, K₂ is an electrode constant corresponding to an electrode model, K₃ is a temperature compensation coefficient, I is the current of the electrode, U is the voltage of the electrode, n is a constant value greater than 0, and A is within 10%-30%.

7. The method for determining a failure cause of a salt chlorinator for a swimming pool according to claim 2, wherein after the outputting the first alarm signal, the following step is performed:
step S311: cleaning the electrode; using acidic solution to clean the electrode to remove dirt on a surface of the electrode; and after cleaning, repeat step S31, and if it is determined that the value is still less than 0, proceeding to step S32.

8. The method for determining a failure cause of a salt chlorinator for a swimming pool according to claim 2, wherein before step S31, the following step is performed:
step S301: determining V; based on an actual volume V of the swimming pool, obtaining a model of a currently working electrode, looking up a used volume threshold V_{threshold} corresponding to the electrode model, and determining whether V is less than or equal to V_{threshold}; and
if V is less than or equal to V_{threshold}, determining that the electrode model is selected properly, and proceeding to step S302; or
if V is not less than or equal to V_{threshold}, determining that the electrode model is not selected properly, outputting a third alarm signal; and
step S302: determining Pec and t; obtaining a percentage Pec of a chlorine production yield and actual working time t set by the user, and performing calculation based on f(Pec, t)=Pec×t and determining whether a value thereof is greater than 1; and
if the value is greater than 1, determining that Pec and t are set properly, and proceeding to step S31; or
if the value is not greater than 1, determining that Pec and t are not set properly, and outputting a fourth alarm signal.

9. The method for determining a failure cause of a salt chlorinator for a swimming pool according to claim 2, wherein before step S32, the following step is performed:
step S312: determining Fₙ, detecting an actual salt concentration Fₙ in the swimming pool, and then determining whether 2,300 ppm≤Fₙ≤6,000 ppm is established; and
if 2,300 ppm≤Fₙ≤6,000 ppm is established, determining that Fₙ added by the user is proper, and proceeding to step S32; or
if 2,300 ppm≤Fₙ≤6,000 ppm is not established, determining that Fₙ added by the user is not proper, and outputting a fifth alarm signal.

10. The method for determining a failure cause of a salt chlorinator for a swimming pool according to claim 1, wherein step S2 is specifically: based on numerical values of a total phosphorus concentration C_{P} in the swimming pool, a cyanuric acid concentration C_{y} in the swimming pool, pH of swimming pool water, a calcium hardness concentration Ch in the swimming pool, and a total alkali concentration Ct in the swimming pool in the water quality test report, determining whether any one or more of C_{P}≤C_{Pthreshold}, C_{y1}≤C_{y}≤C_{y2}, pH₁≤pH≤pH₂, Ch₁≤Ch≤Ch₂, and Ct₁≤Ct≤Ct₂ are established; and
if any one or more of C_{P}≤C_{Pthreshold}, C_{y1}≤C_{y}≤C_{y2}, pH₁≤pH≤pH₂, Ch₁≤Ch≤Ch₂, and Ct₁≤Ct≤Ct₂ are established, determining that there is no problem with water quality, and proceeding to step S3 to continuously determine whether there is a problem with the salt chlorinator; or
if any one or more of C_{P}≤C_{Pthreshold}, C_{y1}≤C_{y}≤C_{y}, pH₁≤pH≤pH₂, Ch₁≤Ch≤Ch₂, and Ct₁≤Ct≤Ct₂ are not established, determining that there is a problem with water quality, and outputting a sixth alarm signal.

11. The method for determining a failure cause of a salt chlorinator for a swimming pool according to claim 1, wherein step S3 comprises the following steps:
determining electrode life; based on an actual salt concentration Fₙ in the swimming pool and a current I and voltage U of an electrode, performing calculation based on an electrode life determining formula f(Fₙ, I, U) and determining whether a value thereof is less than a threshold A; and
if the value is less than the threshold A, determining that the electrode life is not exhausted, and determining whether there is another problem with the salt chlorinator; or
if the value is not less than the threshold A, determining that the electrode life is to be exhausted, outputting a second alarm signal; and
determining whether there is the posed problem; based on a percentage Pec of a chlorine production yield, actual working time t, and the current I of the electrode set by a user, performing calculation based on a posed problem determining formula f(I, Pec, t) and determining whether a value thereof is greater than 0;
if the value is greater than 0, determining whether there is other problem with the salt chlorinator; or
if the value is not greater than 0, outputting the first alarm signal.
